(19) United States
(12) Patent Application Publication
(10) Pub. No.: US 2023/0190904 A1
(43) Pub. Date: Jun. 22, 2023

(54) Compositions and Methods for Treating Morphine, Heroin, and Alcohol Dependence

(71) Applicant: Babak Behnam, Clarksburg, MD (US)

(72) Inventors: Babak Behnam, Clarksburg, MD (US); Azim Akbarzadehkhiavi, Tehran (IR); Afshin Khavari, Tehran (IR)

(73) Assignee: Babak Behnam, Clarksburg, MD (US)

(21) Appl. No.: 17/999,434

(22) PCT Filed: May 26, 2021
(86) PCT No.: PCT/US2021/034197

§ 371 Date: Nov. 21, 2022

Related U.S. Application Data
(60) Provisional application No. 63/030,356, filed on May 27, 2020.

(51) Int. Cl. A61K 39/00; A61K 47/64

(52) U.S. Cl. CPC A61K 39/0013; A61K 47/643; A61K 47/646; A61K 2039/55505

(57) ABSTRACT

This invention provides a composition of matter comprising a plurality of morphine-6-succinyl-BSA, wherein the average ratio of morphine-6-succinyl moieties to BSA is at least 7.0. This invention also provides a composition of matter comprising a plurality of heroin-6-succinyl-B SA, wherein the average ratio of heroin-6-succinyl moieties to BSA is at least 7.0. This invention further provides a composition of matter comprising a plurality of ethanol-succinyl-B SA, wherein the average ratio of ethanol-succinyl moieties to BSA is at least 7.0. This invention still further provides related pharmaceutical compositions, therapeutic methods, prophylactic methods, synthetic methods, and articles of manufacture.

This application claims the benefit of U.S. Provisional Application No. 63/030,356, filed May 27, 2020, the contents of which are incorporated herein by reference.

Throughout this application, various publications are cited. The disclosure of these publications is hereby incorporated by reference into this application to describe more fully the state of the art to which this invention pertains.

### BACKGROUND OF THE INVENTION

Morphine

Morphine is a pain medication of the opiate family, and is found naturally in a number of plants and animals. It acts on the central nervous system (CNS) to decrease pain. It is used both therapeutically and recreationally. It is well known that morphine is highly addictive.

Depending on the method of administration, most of the effects of morphine start to take place within 15-60 minutes and may last for four to six hours. Some possible immediate side effects of morphine use include the following: inability to concentrate; itchy skin; chest pain; drowsiness; slowed breathing; rapid heartbeat; severe respiratory depression; coma; hallucinations; relaxed and calm feeling; sleepiness; dizziness; decreased sexual drive/performance; dry mouth; nervousness; apathy; nausea; mood changes; false sense of well-being; and euphoria.

As a CNS depressant, morphine slows down activity in the brain and nervous system, which can result in slowed breathing and extreme drowsiness. The effects of sedation can be so intense that at high doses, a person may become unconscious.

Prolonged morphine use can lead to many negative side effects, not the least of which is addiction. Other harmful effects include damage to veins at injection sites and substance-induced mood disorders like depression. Long-term side effects of morphine use include the following: depression; suppressed immune system; restlessness; severe constipation; collapsed veins; and confusion.

Morphine tolerance develops quickly, and the user needs to take increasingly higher doses to feel the same effects. People with a tolerance to morphine usually experience symptoms of withdrawal when they attempt to quit or cut back.

Morphine withdrawal can be very uncomfortable, especially for heavy users. Symptoms vary in intensity depending on the user's tolerance, overall health and metabolism, as well as the frequency and duration of drug use. In general, consuming high doses of morphine and abusing the drug for long periods of time lead to more severe symptoms. Common morphine withdrawal symptoms include the following: runny nose; vomiting; diarrhea; bodily aches and pains; excessive sweating; nausea; headaches; sweating; watery eyes; increased blood pressure; agitation; fever; depression; disorientation; insomnia; chills; rapid heartbeat; anxiety; irritability; and muscle aches.

Heroin

Heroin, also known as diamorphine, is a member of the opiate family. It is used primarily as a recreational drug, and is most commonly injected. It also has certain therapeutic uses outside the United States. Heroin is metabolized in the brain to form 6-monoacetylmorphine and morphine, and provides a euphoric effect.

It is well known that heroin is highly addictive, and that cessation of heroin use in addicted subjects is accompanied by withdrawal symptoms. These withdrawal symptoms include, for example, the following: respiratory depression; dry mouth; drowsiness; impaired mental function; constipation; abscesses; infected heart valves;

blood-borne infections; and pneumonia.

Addiction can be treated with behavioral therapy and medications such as buprenorphine, methadone, and naltrexone. A heroin overdose can, of course, be fatal, yet overdoses are otherwise treatable with naloxone.

Alcohol

Alcohol, i.e., ethanol, is an addictive and psychoactive drug, and is one of the oldest and most common recreational substances. It produces “drunkenness”, a mood lift, euphoria, decreased anxiety, increased sociability, and sedation. It also impairs cognition, memory, motor function, and sensory function, and causes generalized depression of central nervous system function.

Alcohol's short-term adverse effects include generalized impairment of neurocognitive function, dizziness, nausea, vomiting, and hangover-like symptoms. Its long-term adverse effects include, for instance, liver damage and brain damage. Alcohol dependence is treatable with behavioral therapy and with certain medications (e.g., naltrexone, acamprosate, and disulfiram).

### SUMMARY OF THE INVENTION

This invention provides a first composition of matter (“first composition”) comprising a plurality of morphine-6-succinyl-bovine serum albumin (BSA), wherein the average ratio of morphine-6-succinyl moieties to BSA is at least 7.0.

This invention also provides a composition of matter (“first pharmaceutical composition”) comprising a plurality of morphine-6-succinyl-BSA, wherein (i) the average ratio of morphine-6-succinyl moieties to BSA is at least 7.0; (ii) the composition is free of EDAC; (iii) the morphine-6-succinyl-BSA is adsorbed onto a suitable adjuvant; and (iv) the morphine-6-succinyl-BSA so adsorbed is suspended in a suitable buffer.

This invention further provides a method (“first therapeutic method”) for treating a subject addicted to morphine comprising administering to the subject an effective amount of the first pharmaceutical composition (i.e., a therapeutically effective amount).

This invention also provides a method (“first prophylactic method”) for inhibiting the onset of morphine addiction in a non-addicted subject comprising administering to the subject an effective amount of the first composition (i.e., a prophylactically effect amount).

This invention provides a first synthetic method for making morphine-6-succinyl-BSA, comprising the following steps:
(a) contacting morphine with succinic anhydride in the presence of benzene under conditions permitting the formation of morphine-6-succinate; and
(b) contacting the resulting morphine-6-succinate with BSA under conditions permitting the formation of morphine-6-succinyl-BSA, wherein (i) such conditions comprise the presence of tributylamine, dioxane, and isobutyl chloroformate, and (ii) neither step (a) nor step (b) is performed in the presence of EDAC.

This invention provides a second synthetic method for making morphine-6-succinyl-BSA, comprising contacting morphine-6-succinate with BSA under conditions permitting the formation of morphine-6-succinyl-BSA, wherein (i) such conditions comprise the presence of tributylamine, dioxane, and isobutyl chloroformate, and (ii) the contacting is performed in the absence of EDAC.

This invention provides first and second articles of manufacture (also referred to as kits). The first article comprises (a) a composition of matter comprising a plurality of morphine-6-succinyl-BSA, wherein (i) the average ratio of morphine-6-succinyl moieties to BSA is at least 7.0; (ii) the composition is free of EDAC; (iii) the morphine-6-succinyl-BSA is adsorbed onto a suitable adjuvant; and (iv) the morphine-6-succinyl-BSA so adsorbed is suspended in a suitable buffer, and (b) a label indicating a use for the composition in treating a human subject addicted to morphine.

The second article comprises (a) a composition of matter comprising a plurality of morphine-6-succinyl-BSA, wherein (i) the average ratio of morphine-6-succinyl moieties to BSA is at least 7.0; (ii) the composition is free of EDAC; (iii) the morphine-6-succinyl-BSA is adsorbed onto a suitable adjuvant; and (iv) the morphine-6-succinyl-BSA so adsorbed is suspended in a suitable buffer, and (b) a label indicating a use for the composition in inhibiting the onset of morphine addiction in a non-addicted human subject.

This invention provides a second composition of matter (“second composition”) comprising a plurality of heroin-6-succinyl-BSA, wherein the average ratio of heroin-6-succinyl moieties to BSA is at least 7.0.

This invention also provides a composition of matter (“second pharmaceutical composition”) comprising a plurality of heroin-6-succinyl-BSA, wherein (i) the average ratio of heroin-6-succinyl moieties to BSA is at least 7.0; (ii) the composition is free of EDAC; (iii) the heroin-6-succinyl-BSA is adsorbed onto a suitable adjuvant; and (iv) the heroin-6-succinyl-BSA so adsorbed is suspended in a suitable buffer.

This invention further provides a method (“second therapeutic method”) for treating a subject addicted to heroin comprising administering to the subject an effective amount of the second pharmaceutical composition (i.e., a therapeutically effective amount).

This invention also provides a method (“second prophylactic method”) for inhibiting the onset of heroin addiction in a non-addicted subject comprising administering to the subject an effective amount of the second composition (i.e., a prophylactically effect amount).

This invention provides a first synthetic method for making heroin-6-succinyl-BSA, comprising the following steps:
(a) contacting heroin with succinic anhydride in the presence of benzene under conditions permitting the formation of heroin-6-succinate; and
(b) contacting the resulting heroin-6-succinate with BSA under conditions permitting the formation of heroin-6-succinyl-BSA, wherein (i) such conditions comprise the presence of tributylamine, dioxane, and isobutyl chloroformate, and (ii) neither step (a) nor step (b) is performed in the presence of EDAC.

This invention provides a second synthetic method for making heroin-6-succinyl-BSA, comprising contacting heroin-6-succinate with BSA under conditions permitting the formation of heroin-6-succinyl-BSA, wherein (i) such conditions comprise the presence of tributylamine, dioxane, and isobutyl chloroformate, and (ii) the contacting is performed in the absence of EDAC.

This invention provides third and fourth articles of manufacture (also referred to as kits). The third article comprises (a) a composition of matter comprising a plurality of heroin-6-succinyl-BSA, wherein (i) the average ratio of heroin-6-succinyl moieties to BSA is at least 7.0; (ii) the composition is free of EDAC; (iii) the heroin-6-succinyl-BSA is adsorbed onto a suitable adjuvant; and (iv) the heroin-6-succinyl-BSA so adsorbed is suspended in a suitable buffer, and (b) a label indicating a use for the composition in treating a human subject addicted to heroin.

The fourth article comprises (a) a composition of matter comprising a plurality of heroin-6-succinyl-BSA, wherein (i) the average ratio of heroin-6-succinyl moieties to BSA is at least 7.0; (ii) the composition is free of EDAC; (iii) the heroin-6-succinyl-BSA is adsorbed onto a suitable adjuvant; and (iv) the heroin-6-succinyl-BSA so adsorbed is suspended in a suitable buffer, and (b) a label indicating a use for the composition in inhibiting the onset of heroin addiction in a non-addicted human subject.

This invention provides a third composition of matter (“third composition”) comprising a plurality of ethanol-succinyl-bovine serum albumin (BSA), wherein the average ratio of ethanol-succinyl moieties to BSA is at least 7.0.

This invention also provides a composition of matter (“third pharmaceutical composition”) comprising a plurality of ethanol-succinyl-BSA, wherein (i) the average ratio of ethanol-succinyl moieties to BSA is at least 7.0; (ii) the composition is free of EDAC; (iii) the ethanol-succinyl-BSA is adsorbed onto a suitable adjuvant; and (iv) the ethanol-succinyl-BSA so adsorbed is suspended in a suitable buffer.

This invention further provides a method (“third therapeutic method”) for treating a subject addicted to alcohol comprising administering to the subject an effective amount of the third pharmaceutical composition (i.e., a therapeutically effective amount).

This invention also provides a method (“third prophylactic method”) for inhibiting the onset of alcohol addiction in a non-addicted subject comprising administering to the subject an effective amount of the third composition (i.e., a prophylactically effect amount).

This invention provides a first synthetic method for making ethanol-succinyl-BSA, comprising the following steps:
(a) contacting ethanol with succinic anhydride in the presence of benzene under conditions permitting the formation of ethanol-succinate; and
(b) contacting the resulting ethanol-succinate with BSA under conditions permitting the formation of ethanol-succinyl-BSA, wherein (i) such conditions comprise the presence of tributylamine, dioxane, and isobutyl chloroformate, and (ii) neither step (a) nor step (b) is performed in the presence of EDAC.

This invention provides a second synthetic method for making ethanol-succinyl-BSA, comprising contacting ethanol-succinate with BSA under conditions permitting the formation of ethanol-succinyl-BSA, wherein (i) such conditions comprise the presence of tributylamine, dioxane, and isobutyl chloroformate, and (ii) the contacting is performed in the absence of EDAC.

This invention provides fifth and sixth articles of manufacture (also referred to as kits). The fifth article comprises (a) a composition of matter comprising a plurality of ethanol-succinyl-BSA, wherein (i) the average ratio of ethanol-succinyl moieties to BSA is at least 7.0; (ii) the composition is free of EDAC; (iii) the ethanol-succinyl-BSA is adsorbed onto a suitable adjuvant; and (iv) the ethanol-succinyl-BSA so adsorbed is suspended in a suitable buffer, and (b) a label indicating a use for the composition in treating a human subject addicted to alcohol.

The sixth article comprises (a) a composition of matter comprising a plurality of ethanol-succinyl-BSA, wherein (i) the average ratio of ethanol-succinyl moieties to BSA is at least 7.0; (ii) the composition is free of EDAC; (iii) the ethanol-succinyl-BSA is adsorbed onto a suitable adjuvant; and (iv) the ethanol-succinyl-BSA so adsorbed is suspended in a suitable buffer, and (b) a label indicating a use for the composition in inhibiting the onset of alcohol addiction in a non-addicted human subject.

### BRIEF DESCRIPTION OF THE FIGURES

FIG. 1 This figure shows the synthesis of pure morphine.

FIG. 2 This figure shows the synthesis of morphine-6-succinate (M-6-S).

FIG. 3 This figure shows the synthesis of morphine-6-succinyl-BSA (M-6-S-BSA). The role of the dioxane, triethylamine and isobutyl chloroformate in this reaction is to bind molecules of morphine succinate to BSA.

FIG. 4 This figure shows a schematic of eight molecules of morphine succinate conjugated with bovine serum albumin to form a morphine vaccine.

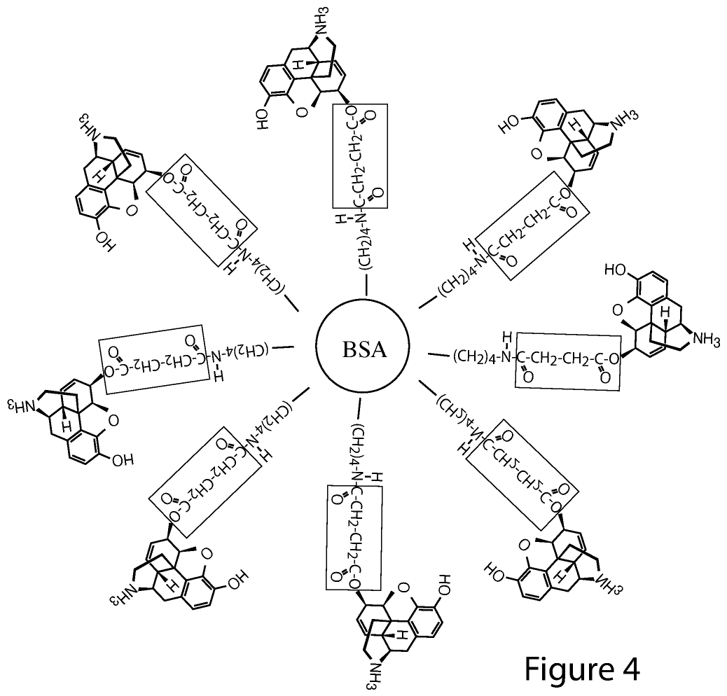

FIG. 5 This figure shows the synthesis of heroin-6-succinyl-BSA (H-6-S-BSA). In this reaction, morphine is first converted to heroin-6-succinate by anhydride acetic acid. It is then converted to 3- and 6-acetyl heroin by anhydride acetic acid. The dioxane, triethylamine and isobutyl chloroformate in this reaction binds eight molecules of heroin succinate to BSA.

FIG. 6 This figure shows a schematic of eight molecules of heroin succinate conjugated with bovine serum albumin to form a heroin vaccine.

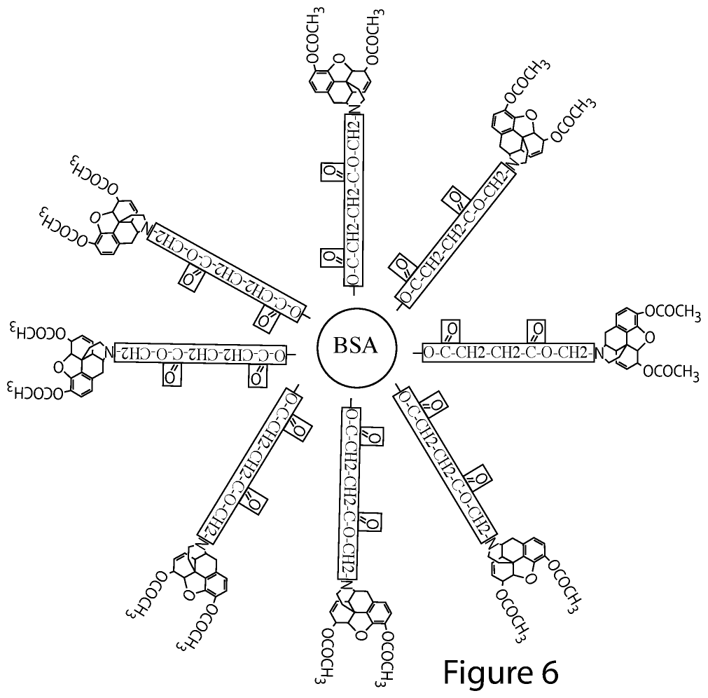

FIG. 7 This figure shows the synthesis of ethanol-succinate and ethanol-succinyl-BSA (E-S-BSA).

FIG. 8 This figure shows a schematic of eight molecules of ethanol succinate conjugated with bovine serum albumin to form an alcohol vaccine.

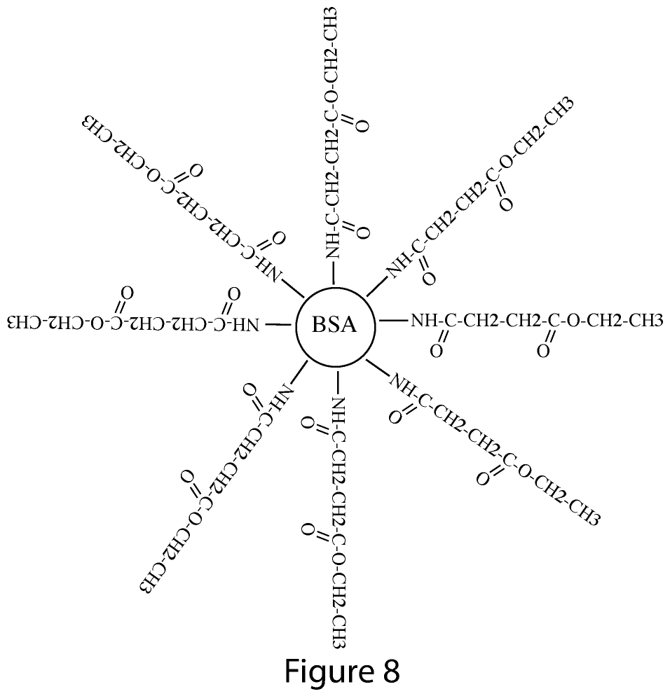

### DETAILED DESCRIPTION OF THE INVENTION

Definitions

In this application, certain terms are used which shall have the meanings set forth as follows.

As used herein, “administer”, with respect to an agent, means to deliver the agent to a subject's body via any known method. Specific modes of administration include, without limitation, intravenous, intramuscular, oral, sublingual, transdermal, subcutaneous, intraperitoneal, and intrathecal administration. Preferred in this invention is intramuscular administration (e.g., injection into the deltoid muscle).

In addition, in this invention, the various agents (e.g., morphine-6-succinyl-BSA, heroin-6-succinyl-BSA, and ethanol-succinyl-BSA) used can be formulated using one or more routinely used pharmaceutically acceptable carriers appropriate for protein agents. Such carriers are well known to those skilled in the art. For example, injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone. Also included in injectable drug delivery systems are adjuvants (e.g., analgesic adjuvants; inorganic compounds (e.g., alum, aluminum hydroxide, aluminum phosphate, and calcium phosphate); mineral oil (e.g., paraffin oil); bacterial products (killed bacteria Bordetella pertussis, Mycobacterium bovis, toxoids; nonbacterial organics (e.g., squalene); plant saponins from Quillaja, soybean, polygala senega; cytokines (e.g., IL-1, IL-2, and IL-12); Freund's complete adjuvant; and Freund's incomplete adjuvant) and buffers (e.g., phosphate buffered saline) or other diluents such as sterile water.

As used herein, an “effective amount” of the first pharmaceutical composition used in the first prophylactic and therapeutic methods is an amount sufficient to deliver to the subject a prophylactic or therapeutic amount of the morphine-6-succinyl-BSA (“active agent”) therein, as appropriate. In one embodiment, an effective amount of the first pharmaceutical composition contains an amount of active agent sufficient to deliver from 1 μg to 1,000 μg of active agent to the subject per dose (e.g., 1 μg, 2.5 μg, 5 μg, 7.5 μg, 10 μg, 12.5 μg, 15 μg, 20 μg, 50 μg, 100 μg, 200 μg, 300 μg, 400 μg, 500 μg, 600 μg, 700 μg, 800 μg, 900 μg, or 1,000 μg; or 1-10 μg, 10-20 μg, 20-50 μg, 50-100 pg, 100-500 μg, or 500-1,000 μg). Moreover, this dose can be administered once, or preferably a plurality of times (ideally three) over the course of prophylaxis or therapy. Similarly, as used herein, an “effective amount” of the second pharmaceutical composition used in the second prophylactic and therapeutic methods is an amount sufficient to deliver to the subject a prophylactic or therapeutic amount of the heroin-6-succinyl-BSA (“active agent”) therein, as appropriate. In one embodiment, an effective amount of the second pharmaceutical composition contains an amount of active agent sufficient to deliver from 1 μg to 1,000 μg of active agent to the subject per dose (e.g., 1 μg, 2.5 μg, 5 μg, 7.5 μg, 10 μg, 12.5 μg, 15 μg, 20 μg, 50 μg, 100 μg, 200 μg, 300 μg, 400 μg, 500 μg, 600 μg, 700 μg, 800 μg, 900 μg, or 1,000 μg; or 1-10 μg, 10-20 μg, 20-50 μg, 50-100 μg, 100-500 μg, or 500-1,000 μg). Moreover, this dose can be administered once, or preferably a plurality of times (ideally three) over the course of prophylaxis or therapy. Similarly, as used herein, an “effective amount” of the third pharmaceutical composition used in the third prophylactic and therapeutic methods is an amount sufficient to deliver to the subject a prophylactic or therapeutic amount of the ethanol-succinyl-BSA (“active agent”) therein, as appropriate. In one embodiment, an effective amount of the third pharmaceutical composition contains an amount of active agent sufficient to deliver from 1 μg to 1,000 μg of active agent to the subject per dose (e.g., 1 μg, 2.5 μg, 5 μg, 7.5 μg, 10 μg, 12.5 μg, 15 μg, 20 μg, 50 μg, 100 μg, 200 μg, 300 μg, 400 μg, 500 μg, 600 μg, 700 μg, 800 μg, 900 μg, or 1,000 μg; or 1-10 μg, 10-20 μg, 20-50 μg, 50-100 μg, 100-500 μg, or 500-1,000 μg). Moreover, this dose can be administered once, or preferably a plurality of times (ideally three) over the course of prophylaxis or therapy.

As used herein, a composition is “free of” EDAC if, for example, it contains less than 1.0% of EDAC, less than 0.9% of EDAC, less than 0.8% of EDAC, less than 0.7% of EDAC, less than 0.6% of EDAC, less than 0.5% of EDAC, less than 0.4% of EDAC, less than 0.3% of EDAC, less than 0.2% of EDAC, less than 0.1% of EDAC, less than 0.05% of EDAC, less than 0.04% of EDAC, less than 0.03% of EDAC, less than 0.02% of EDAC, less than 0.01% of EDAC, or less than 0.001% of EDAC. If a reaction step is not “performed in the presence of EDAC”, the conditions for performing that step are “free of” EDAC. Similarly, a composition is “free of” any amidine if, for example, it contains less than 1.0% of any amidine, less than 0.9% of any amidine, less than 0.8% of any amidine, less than 0.7% of any amidine, less than 0.6% of any amidine, less than 0.5% of any amidine, less than 0.4% of any amidine, less than 0.3% of any amidine, less than 0.2% of any amidine, less than 0.1% of any amidine, less than 0.05% of any amidine, less than 0.04% of any amidine, less than 0.03% of any amidine, less than 0.02% of any amidine, less than 0.01% of any amidine, or less than 0.001% of any amidine. If a reaction step is not “performed in the presence an amidine”, the conditions for performing that step are “free of” any amidine.

As used herein, a “non-addicted” subject, with respect to morphine, includes, without limitation, (i) a subject who has never taken morphine, and (ii) a subject who has taken morphine previously (e.g., medically or recreationally) yet is not addicted (as that term is defined herein). Similarly, as used herein, a “non-addicted” subject, with respect to heroin, includes, without limitation, (i) a subject who has never taken heroin, and (ii) a subject who has taken heroin previously (e.g., medically or recreationally) yet is not addicted (as that term is defined herein). Similarly, as used herein, a “non-addicted” subject, with respect to alcohol, includes, without limitation, (i) a subject who has never consumed alcohol, and (ii) a subject who has consumed alcohol previously (e.g., medically or recreationally) yet is not addicted (as that term is defined herein).

As used herein, a “human subject” can be of any age, gender, or state of co-morbidity. In one embodiment, the subject is male, and in another, the subject is female.

As used herein, a morphine²addicted″ subject is one who craves morphine and, if presented with an opportunity to take morphine, is incapable of resisting that opportunity absent medical or therapeutic intervention. Similarly, as used herein, a heroin-“addicted” subject is one who craves heroin and, if presented with an opportunity to take heroin, is incapable of resisting that opportunity absent medical or therapeutic intervention. Similarly, as used herein, an alcohol-“addicted” subject is one who craves alcohol (i.e., ethanol) and, if presented with an opportunity to consume alcohol, is incapable of resisting that opportunity absent medical or therapeutic intervention.

As used herein, “inhibiting the onset” of morphine addiction in a non-addicted subject includes, without limitation, reducing the likelihood of addiction by at least 10%, at least 20%, at least 30%, at least 40%, at least 50%, at least 60%, at least 70%, at least 80%, at least 90%, or at least 95%. Preferably, inhibiting the onset of morphine addiction in a non-addicted subject means preventing such onset. Similarly, as used herein, “inhibiting the onset” of heroin addiction in a non-addicted subject includes, without limitation, reducing the likelihood of addiction by at least 10%, at least 20%, at least 30%, at least 40%, at least 50%, at least 60%, at least 70%, at least 80%, at least 90%, or at least 95%. Preferably, inhibiting the onset of heroin addiction in a non-addicted subject means preventing such onset. Similarly, as used herein, “inhibiting the onset” of alcohol addiction in a non-addicted subject includes, without limitation, reducing the likelihood of addiction by at least 10%, at least 20%, at least 30%, at least 40%, at least 50%, at least 60%, at least 70%, at least 80%, at least 90%, or at least 95%. Preferably, inhibiting the onset of alcohol addiction in a non-addicted subject means preventing such onset.

As used herein, the term “subject” includes, without limitation, a mammal such as a human, a non-human primate, a dog, a cat, a horse, a sheep, a goat, a cow, a rabbit, a pig, a hamster, a rat and a mouse.

As used herein, “treating” a subject addicted to morphine and symptomatic of that addiction includes, without limitation, (i) slowing, stopping or reversing the progression of one or more symptoms of addiction, (ii) reducing or eliminating the addiction, (iii) reducing or eliminating the likelihood of the symptoms' recurrence, and/or (iv) reducing the likelihood of, or preventing, the addiction's recurrence. In the preferred embodiment, treating a subject addicted to morphine and symptomatic of that addiction includes (i) reversing the progression of one or more of the symptoms, (ii) eliminating the addiction, (iii) preventing the symptoms' recurrence, and/or (iv) preventing the addiction's recurrence. The progress of treating a subject addicted to morphine and symptomatic of that addiction can be measured according to a number of clinical endpoints. These endpoints include, without limitation, amelioration of one or more of the following: dilated pupils; nodding off; slurred speech; inattention; shallow breathing; neglecting daily responsibilities; isolation from loved ones; irritability; and mood swings.

Similarly, as used herein, “treating” a subject addicted to heroin and symptomatic of that addiction includes, without limitation, (i) slowing, stopping or reversing the progression of one or more symptoms of addiction, (ii) reducing or eliminating the addiction, (iii) reducing or eliminating the likelihood of the symptoms' recurrence, and/or (iv) reducing the likelihood of, or preventing, the addiction's recurrence. In the preferred embodiment, treating a subject addicted to heroin and symptomatic of that addiction includes (i) reversing the progression of one or more of the symptoms, (ii) eliminating the addiction, (iii) preventing the symptoms' recurrence, and/or (iv) preventing the addiction's recurrence. The progress of treating a subject addicted to heroin and symptomatic of that addiction can be measured according to a number of clinical endpoints. These endpoints include, without limitation, amelioration of one or more of the following: respiratory depression; dry mouth; drowsiness; impaired mental function; constipation: abscesses: heart valve infection: blood-borne infections; and pneumonia

Similarly, as used herein, “treating” a subject addicted to alcohol and symptomatic of that addiction includes, without limitation, (i) slowing, stopping or reversing the progression of one or more symptoms of addiction, (ii) reducing or eliminating the addiction, (iii) reducing or eliminating the likelihood of the symptoms' recurrence, and/or (iv) reducing the likelihood of, or preventing, the addiction's recurrence. In the preferred embodiment, treating a subject addicted to alcohol and symptomatic of that addiction includes (i) reversing the progression of one or more of the symptoms, (ii) eliminating the addiction, (iii) preventing the symptoms' recurrence, and/or (iv) preventing the addiction's recurrence. The progress of treating a subject addicted to alcohol and symptomatic of that addiction can be measured according to a number of clinical endpoints. These endpoints include, without limitation, amelioration of one or more of the following: impaired cognition; impaired memory; impaired motor function; impaired sensory function; generalized depression of central nervous system function; generalized impairment of neurocognitive function; liver damage; and brain damage.

Embodiments of the Invention

This application provides compositions having use as vaccines for patients dependent on morphine, heroin, or alcohol, or at risk of becoming dependent thereon. These compositions are unexpectedly superior because, among other reasons, they separately comprise morphine-6-succinyl-BSA, heroin-6-succinyl-BSA, and ethanol-succinyl-BSA having advantageously high ratios of hapten to BSA, and are free of EDAC and other harmful amidines.

Morphine-6-Succinyl-BSA

Specifically, this invention provides a first composition of matter (“first composition”) comprising a plurality of morphine-6-succinyl-BSA, wherein the average ratio of morphine-6-succinyl moieties to BSA is at least 7.0.

In one embodiment, the first composition is free of the amidine 1-ethyl-3-(3-dimethylaminopropyl) carbodiimide (EDAC). Preferably, the first composition is also free of any amidine.

In one embodiment of the first composition, the average ratio of morphine-6-succinyl moieties to BSA is selected from the group consisting of at least 7.5, at least 8.0, and preferably at least 8.2. By way of example, in the first composition, the average ratio of morphine-6-succinyl moieties to BSA is at least one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the first composition, the average ratio of morphine-6-succinyl moieties to BSA is one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the first composition, the average ratio of morphine-6-succinyl moieties to BSA is one of the following: 7.0-7.1; 7.1-7.2; 7.2-7.3; 7.3-7.4; 7.4-7.5; 7.5-7.6; 7.6-7 .7; 7 .7-7.8; 7.8-7.9; 7.9-8.0; 8.0-8.1; 8.1-8.2; 8.2-8.3; 8.3-8.4; 8.4-8.5; 8.5-8.6; 8.6-8.7; 8.7-8.8; 8.8-8.9; 8.9-9.0; 9.0-9.1; 9.1-9.2; 9.2-9.3; 9.3-9.4; 9.4-9.5; 9.5-9.6; 9.6-9.7; 9.7-9.8; 9.8-9.9; or 9.9-10.0; 7.0-7.2; 7.2-7.4; 7.4-7.6; 7.6-7.8; 7.8-8.0; 8.0-8.2; 8.2-8.4; 8.4-8.6; 8.6-8.8; 8.8-9.0; 9.0-9.2; 9.2-9.4; 9.4-9.6; 9.6-9.8; or 9.8-10.0.

In a preferred embodiment, the first composition comprises a plurality of morphine succinyl-BSA, wherein (i) the average ratio of morphine-6-succinyl moieties to BSA is at least 8.2; and (ii) the composition is free of EDAC (and is preferably free of any am idine).

This invention also provides a composition of matter (“first pharmaceutical composition”) comprising a plurality of morphine-6-succinyl-BSA, wherein (i) the average ratio of morphine-6-succinyl moieties to BSA is at least 7.0; (ii) the composition is free of EDAC; (iii) the morphine-6-succinyl-BSA is adsorbed onto a suitable adjuvant; and (iv) the morphine-6-succinyl-BSA so adsorbed is suspended in a suitable buffer. Preferably, the composition is also free of any amidine.

In one embodiment of the first pharmaceutical composition, the average ratio of morphine-6-succinyl moieties to BSA is selected from the group consisting of at least 7.5, at least 8.0, and preferably at least 8.2. By way of example, in the first pharmaceutical composition, the average ratio of morphine-6-succinyl moieties to BSA is at least one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the first pharmaceutical composition, the average ratio of morphine-6-succinyl moieties to BSA is one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the first pharmaceutical composition, the average ratio of morphine-6-succinyl moieties to BSA is one of the following: 7.0-7.1; 7.1-7.2; 7.2-7.3; 7.3-7.4; 7.4-7.5; 7.5-7.6; 7.6-7.7; 7.7-7.8; 7.8-7.9; 7.9-8.0; 8.0-8.1; 8.1-8.2; 8.2-8.3; 8.3-8.4; 8.4-8.5; 8.5-8.6; 8.6-8.7; 8.7-8.8; 8.8-8.9; 8.9-9.0; 9.0-9.1; 9.1-9.2; 9.2-9.3; 9.3-9.4; 9.4-9.5; 9.5-9.6; 9.6-9.7; 9.7-9.8; 9.8-9.9; or 9.9-10.0; 7.0-7.2; 7.2-7.4; 7.4-7.6; 7.6-7.8; 7.8-8.0; 8.0-8.2; 8.2-8.4; 8.4-8.6; 8.6-8.8; 8.8-9.0; 9.0-9.2; 9.2-9.4; 9.4-9.6; 9.6-9.8; or 9.8-10.0.

Adjuvants suitable for use in the first pharmaceutical composition are known. In a preferred embodiment, the suitable adjuvant is aluminum hydroxide gel. Similarly, buffers suitable for use in the first pharmaceutical composition are known. In a preferred embodiment, the suitable buffer is phosphate buffered saline.

In a preferred embodiment, the first pharmaceutical composition comprises a plurality of morphine-6-succinyl-BSA, wherein (i) the average ratio of morphine-6-succinyl moieties to BSA is at least 8.2; (ii) the composition is free of EDAC (and preferably is free of any amidine); (iii) the morphine-6-succinyl-BSA is adsorbed onto aluminum hydroxide gel; and (iv) the morphine-6-succinyl-BSA so adsorbed is suspended in phosphate buffered saline.

This invention further provides a method (“first therapeutic method”) for treating a subject addicted to morphine comprising administering to the subject an effective amount of the first pharmaceutical composition (i.e., a therapeutically effective amount). Preferably, the subject is human.

In the first therapeutic method, the first pharmaceutical composition can be administered according to any appropriate dosing regimen. In one embodiment, the first pharmaceutical composition is administered via a single dose. In a preferred embodiment, the composition is administered via a plurality of doses, such as two doses 30 days apart. In a further preferred embodiment, the composition is administered via three doses at 30-day intervals (i.e., at day 0, day 30 and day 60). In yet another embodiment, this administration is followed with one or more “boosters” at suitable intervals (e.g., every two or three years).

In a preferred embodiment of the first therapeutic method, the method comprises injecting the subject with an effective amount of the first pharmaceutical composition, wherein the composition is administered via three doses at 30-day intervals. Preferably, the subject is human.

This invention also provides a method (“first prophylactic method”) for inhibiting the onset of morphine addiction in a non-addicted subject comprising administering to the subject an effective amount of the first composition (i.e., a prophylactically effect amount). Preferably, the subject is human.

In the first prophylactic method, the first pharmaceutical composition can be administered according to any appropriate dosing regimen. In one embodiment, the first pharmaceutical composition is administered via a single dose. In a preferred embodiment, the composition is administered via a plurality of doses, such as two doses 30 days apart. In a further preferred embodiment, the composition is administered via three doses at 30-day intervals (i.e., at day 0, day 30 and day 60). In yet another embodiment, this administration is followed with one or more “boosters” at suitable intervals (e.g., every two or three years).

In a preferred embodiment of the first prophylactic method, the method comprises injecting the human subject with an effective amount of the first prophylactic composition, wherein the composition is administered via three doses at 30-day intervals.

The first composition (as opposed to the first pharmaceutical composition) is also envisioned, mutatis mutandis, for use in the present therapeutic and prophylactic methods.

This invention provides a first synthetic method for making morphine-6-succinyl-BSA, comprising the following steps:
(a) contacting morphine with succinic anhydride in the presence of benzene under conditions permitting the formation of morphine-6-succinate; and
(b) contacting the resulting morphine-6-succinate with BSA under conditions permitting the formation of morphine-6-succinyl-BSA, wherein (i) such conditions comprise the presence of tributylamine, dioxane, and isobutyl chloroformate, and (ii) neither step (a) nor step (b) is performed in the presence of EDAC.

In a preferred embodiment of the first synthetic method, neither step (a) nor step (b) is performed in the presence of an amidine.

This invention provides a second synthetic method for making morphine-6-succinyl-BSA, comprising contacting morphine-6-succinate with BSA under conditions permitting the formation of morphine-6-succinyl-BSA, wherein (i) such conditions comprise the presence of tributylamine, dioxane, and isobutyl chloroformate, and (ii) the contacting is performed in the absence of EDAC.

In a preferred embodiment of the second synthetic method, the contacting is performed in the absence of any amidine.

This invention provides first and second articles of manufacture (also referred to as kits). The first article comprises (a) a composition of matter comprising a plurality of morphine-6-succinyl-BSA, wherein (i) the average ratio of morphine-6-succinyl moieties to BSA is at least 7.0; (ii) the composition is free of EDAC; (iii) the morphine-6-succinyl-BSA is adsorbed onto a suitable adjuvant; and (iv) the morphine-6-succinyl-BSA so adsorbed is suspended in a suitable buffer, and (b) a label indicating a use for the composition in treating a human subject addicted to morphine.

In a preferred embodiment, the first article comprises (a) a composition of matter comprising a plurality of morphine-6-succinyl-BSA, wherein (i) the average ratio of morphine-6-succinyl moieties to BSA is at least 8.2; (ii) the composition is free of EDAC; (iii) the morphine-6-succinyl-BSA is adsorbed onto aluminum hydroxide gel; and (iv) the morphine-6-succinyl-BSA so adsorbed is suspended in phosphate buffered saline, and (b) a label indicating a use for the composition in treating a human subject addicted to morphine.

The second article comprises (a) a composition of matter comprising a plurality of morphine-6-succinyl-BSA, wherein (i) the average ratio of morphine-6-succinyl moieties to BSA is at least 7.0; (ii) the composition is free of EDAC; (iii) the morphine-6-succinyl-BSA is adsorbed onto a suitable adjuvant; and (iv) the morphine-6-succinyl-BSA so adsorbed is suspended in a suitable buffer, and (b) a label indicating a use for the composition in inhibiting the onset of morphine addiction in a non-addicted human subject.

In a preferred embodiment, the second article comprises (a) a composition of matter comprising a plurality of morphine-6-succinyl-BSA, wherein (i) the average ratio of morphine-6-succinyl moieties to BSA is at least 8.2; (ii) the composition is free of EDAC; (iii) the morphine-6-succinyl-BSA is adsorbed onto aluminum hydroxide gel; and (iv) the morphine-6-succinyl-BSA so adsorbed is suspended in phosphate buffered saline, and (b) a label indicating a use for the composition in inhibiting the onset of morphine addiction in a non-addicted human subject.

Preferably, in the first and second articles, the composition is free of any amidine.

Heroin-6-Succinyl-BSA

This invention provides a second composition of matter (“second composition”) comprising a plurality of heroin-6-succinyl-BSA, wherein the average ratio of heroin-6-succinyl moieties to BSA is at least 7.0.

In one embodiment, the second composition is free of the amidine 1-ethyl-3-(3-dimethylaminopropyl) carbodiimide (EDAC). Preferably, the second composition is also free of any amidine.

In one embodiment of the second composition, the average ratio of heroin-6-succinyl moieties to BSA is selected from the group consisting of at least 7.2, at least 7.3, and preferably at least 7.4. By way of example, in the second composition, the average ratio of heroin-6-succinyl moieties to BSA is at least one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the second composition, the average ratio of heroin-6-succinyl moieties to BSA is one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the second composition, the average ratio of heroin-6-succinyl moieties to BSA is one of the following: 7.0-7.1; 7.1-7.2; 7.2-7.3; 7.3-7.4; 7.4-7.5; 7.5-7.6; 7.6-7.7; 7.7-7.8; 7.8-7.9; 7.9-8.0; 8.0-8.1; 8.1-8.2; 8.2-8.3; 8.3-8.4; 8.4-8.5; 8.5-8.6; 8.6-8.7; 8.7-8.8; 8.8-8.9; 8.9-9.0; 9.0-9.1; 9.1-9.2; 9.2-9.3; 9.3-9.4; 9.4-9.5; 9.5-9.6; 9.6-9.7; 9.7-9.8; 9.8-9.9; or 9.9-10.0; 7.0-7.2; 7.2-7.4; 7.4-7.6; 7.6-7.8; 7.8-8.0; 8.0-8.2; 8.2-8.4; 8.4-8.6; 8.6-8.8; 8.8-9.0; 9.0-9.2; 9.2-9.4; 9.4-9.6; 9.6-9.8; or 9.8-10.0.

In a preferred embodiment, the second composition comprises a plurality of heroin-6-succinyl-BSA, wherein (i) the average ratio of heroin-6-succinyl moieties to BSA is at least 7.4; and (ii) the composition is free of EDAC (and is preferably free of any amidine).

This invention also provides a composition of matter (“second pharmaceutical composition”) comprising a plurality of heroin-6-succinyl-BSA, wherein (i) the average ratio of heroin-6-succinyl moieties to BSA is at least 7.5; (ii) the composition is free of EDAC; (iii) the heroin-6-succinyl-BSA is adsorbed onto a suitable adjuvant; and (iv) the heroin-6-succinyl-BSA so adsorbed is suspended in a suitable buffer. Preferably, the composition is also free of any amidine.

In one embodiment of the second pharmaceutical composition, the average ratio of heroin-6-succinyl moieties to BSA is selected from the group consisting of at least 7.2, at least 7.3, and preferably at least 7.4. By way of example, in the second pharmaceutical composition, the average ratio of heroin-6-succinyl moieties to BSA is at least one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the second pharmaceutical composition, the average ratio of heroin-6-succinyl moieties to BSA is one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the second pharmaceutical composition, the average ratio of heroin-6-succinyl moieties to BSA is one of the following: 7.0-7.1; 7.1-7.2; 7.2-7.3; 7.3-7.4; 7.4-7.5; 7.5-7.6; 7.6-7.7; 7.7-7.8; 7.8-7.9; 7.9-8.0; 8.0-8.1; 8.1-8.2; 8.2-8.3; 8.3-8.4; 8.4-8.5; 8.5-8.6; 8.6-8.7; 8.7-8.8; 8.8-8.9; 8.9-9.0; 9.0-9.1; 9.1-9.2; 9.2-9.3; 9.3-9.4; 9.4-9.5; 9.5-9.6; 9.6-9.7; 9.7-9.8; 9.8-9.9; or 9.9-10.0; 7.0-7.2; 7.2-7.4; 7.4-7.6; 7.6-7.8; 7.8-8.0; 8.0-8.2; 8.2-8.4; 8.4-8.6; 8.6-8.8; 8.8-9.0; 9.0-9.2; 9.2-9.4; 9.4-9.6; 9.6-9.8; or 9.8-10.0.

Adjuvants suitable for use in the second pharmaceutical composition are known. In a preferred embodiment, the suitable adjuvant is aluminum hydroxide gel. Similarly, buffers suitable for use in the second pharmaceutical composition are known. In a preferred embodiment, the suitable buffer is phosphate buffered saline.

In a preferred embodiment, the second pharmaceutical composition comprises a plurality of heroin-6-succinyl-BSA, wherein (i) the average ratio of heroin-6-succinyl moieties to BSA is at least 7.4; (ii) the composition is free of EDAC (and preferably is free of any amidine); (iii) the heroin-6-succinyl-BSA is adsorbed onto aluminum hydroxide gel; and (iv) the heroin-6-succinyl-BSA so adsorbed is suspended in phosphate buffered saline.

This invention further provides a method (“second therapeutic method”) for treating a subject addicted to heroin comprising administering to the subject an effective amount of the second pharmaceutical composition (i.e., a therapeutically effective amount). Preferably, the subject is human.

In the second therapeutic method, the second pharmaceutical composition can be administered according to any appropriate dosing regimen. In one embodiment, the second pharmaceutical composition is administered via a single dose. In a preferred embodiment, the composition is administered via a plurality of doses, such as two doses 30 days apart. In a further preferred embodiment, the composition is administered via three doses at 30-day intervals (i.e., at day 0, day 30 and day 60). In yet another embodiment, this administration is followed with one or more boosters at suitable intervals (e.g., every two or three years).

In a preferred embodiment of the second therapeutic method, the method comprises injecting the subject with an effective amount of the second pharmaceutical composition, wherein the composition is administered via three doses at 30-day intervals. Preferably, the subject is human.

This invention also provides a method (“second prophylactic method”) for inhibiting the onset of heroin addiction in a non-addicted subject comprising administering to the subject an effective amount of the second composition (i.e., a prophylactically effect amount). Preferably, the subject is human.

In the second prophylactic method, the second pharmaceutical composition can be administered according to any appropriate dosing regimen. In one embodiment, the second pharmaceutical composition is administered via a single dose. In a preferred embodiment, the composition is administered via a plurality of doses, such as two doses 30 days apart. In a further preferred embodiment, the composition is administered via three doses at 30-day intervals (i.e., at day 0, day 30 and day 60). In yet another embodiment, this administration is followed with one or more “boosters” at suitable intervals (e.g., every two or three years).

In a preferred embodiment of the second prophylactic method, the method comprises injecting the human subject with an effective amount of the second prophylactic composition, wherein the composition is administered via three doses at 30-day intervals.

The second composition (as opposed to the second pharmaceutical composition) is also envisioned, mutatis mutandis, for use in the present therapeutic and prophylactic methods.

This invention provides a first synthetic method for making heroin-6-succinyl-BSA, comprising the following steps:
(a) contacting heroin with succinic anhydride in the presence of benzene under conditions permitting the formation of heroin-6-succinate; and
(b) contacting the resulting heroin-6-succinate with BSA under conditions permitting the formation of heroin-6-succinyl-BSA, wherein (i) such conditions comprise the presence of tributylamine, dioxane, and isobutyl chloroformate, and (ii) neither step (a) nor step (b) is performed in the presence of EDAC.

In a preferred embodiment of the first synthetic method, neither step (a) nor step (b) is performed in the presence of an amidine.

This invention provides a second synthetic method for making heroin-6-succinyl-BSA, comprising contacting heroin-6-succinate with BSA under conditions permitting the formation of heroin-6-succinyl-BSA, wherein (i) such conditions comprise the presence of tributylamine, dioxane, and isobutyl chloroformate, and (ii) the contacting is performed in the absence of EDAC.

In a preferred embodiment of the second synthetic method, the contacting is performed in the absence of any amidine.

This invention provides third and fourth articles of manufacture (also referred to as kits). The third article comprises (a) a composition of matter comprising a plurality of heroin-6-succinyl-BSA, wherein (i) the average ratio of heroin-6-succinyl moieties to BSA is at least 7.0; (ii) the composition is free of EDAC; (iii) the heroin-6-succinyl-BSA is adsorbed onto a suitable adjuvant; and (iv) the heroin-6-succinyl-BSA so adsorbed is suspended in a suitable buffer, and (b) a label indicating a use for the composition in treating a human subject addicted to heroin.

In a preferred embodiment, the third article comprises (a) a composition of matter comprising a plurality of heroin-6-succinyl-BSA, wherein (i) the average ratio of heroin-6-succinyl moieties to BSA is at least 7.4; (ii) the composition is free of EDAC; (iii) the heroin-6-succinyl-BSA is adsorbed onto aluminum hydroxide gel; and (iv) the heroin-6-succinyl-BSA so adsorbed is suspended in phosphate buffered saline, and (b) a label indicating a use for the composition in treating a human subject addicted to heroin.

The fourth article comprises (a) a composition of matter comprising a plurality of heroin-6-succinyl-BSA, wherein (i) the average ratio of heroin-6-succinyl moieties to BSA is at least 7.0; (ii) the composition is free of EDAC; (iii) the heroin-6-succinyl-BSA is adsorbed onto a suitable adjuvant; and (iv) the heroin-6-succinyl-BSA so adsorbed is suspended in a suitable buffer, and (b) a label indicating a use for the composition in inhibiting the onset of heroin addiction in a non-addicted human subject.

In a preferred embodiment, the fourth article comprises (a) a composition of matter comprising a plurality of heroin-6-succinyl-BSA, wherein (i) the average ratio of heroin-6-succinyl moieties to BSA is at least 7.4; (ii) the composition is free of EDAC; (iii) the heroin-6-succinyl-BSA is adsorbed onto aluminum hydroxide gel; and (iv) the heroin-6-succinyl-BSA so adsorbed is suspended in phosphate buffered saline, and (b) a label indicating a use for the composition in inhibiting the onset of heroin addiction in a non-addicted human subject.

Preferably, in the third and fourth articles, the composition is free of any amidine.

Ethanol-Succinyl-BSA

This invention provides a third composition of matter (“third composition”) comprising a plurality of ethanol-succinyl-BSA, wherein the average ratio of ethanol-succinyl moieties to BSA is at least 7.0.

In one embodiment, the third composition is free of the amidine 1-ethyl-3-(3-dimethylaminopropyl) carbodiimide (EDAC). Preferably, the third composition is also free of any amidine.

In one embodiment of the third composition, the average ratio of ethanol-succinyl moieties to BSA is selected from the group consisting of at least 7.3, at least 7.4, and preferably at least 7.5. By way of example, in the third composition, the average ratio of ethanol-succinyl moieties to BSA is at least one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the third composition, the average ratio of ethanol-succinyl moieties to BSA is one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the third composition, the average ratio of ethanol-succinyl moieties to BSA is one of the following: 7.0-7.1; 7.1-7.2; 7.2-7.3; 7.3-7.4; 7.4-7.5; 7.5-7.6; 7.6-7.7; 7.7-7.8; 7.8-7.9; 7.9-8.0; 8.0-8.1; 8.1-8.2; 8.2-8.3; 8.3-8.4; 8.4-8.5; 8.5-8.6; 8.6-8.7; 8.7-8.8; 8.8-8.9; 8.9-9.0; 9.0-9.1; 9.1-9.2; 9.2-9.3; 9.3-9.4; 9.4-9.5; 9.5-9.6; 9.6-9.7; 9.7-9.8; 9.8-9.9; or 9.9-10.0; 7.0-7.2; 7.2-7.4; 7.4-7.6; 7.6-7.8; 7.8-8.0; 8.0-8.2; 8.2-8.4; 8.4-8.6; 8.6-8.8; 8.8-9.0; 9.0-9.2; 9.2-9.4; 9.4-9.6; 9.6-9.8; or 9.8-10.0.

In a preferred embodiment, the third composition comprises a plurality of ethanol-succinyl-BSA, wherein (i) the average ratio of ethanol-succinyl moieties to BSA is at least 7.5; and (ii) the composition is free of EDAC (and is preferably free of any am idine).

This invention also provides a composition of matter (“third pharmaceutical composition”) comprising a plurality of ethanol-succinyl-BSA, wherein (i) the average ratio of ethanol-succinyl moieties to BSA is at least 7.0; (ii) the composition is free of EDAC; (iii) the ethanol-succinyl-BSA is adsorbed onto a suitable adjuvant; and (iv) the ethanol-succinyl-BSA so adsorbed is suspended in a suitable buffer. Preferably, the composition is also free of any amidine.

In one embodiment of the third pharmaceutical composition, the average ratio of ethanol-succinyl moieties to BSA is selected from the group consisting of at least 7.3, at least 7.4, and preferably at least 7.5. By way of example, in the third pharmaceutical composition, the average ratio of ethanol-succinyl moieties to BSA is at least one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the third pharmaceutical composition, the average ratio of ethanol-succinyl moieties to BSA is one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the third pharmaceutical composition, the average ratio of ethanol-succinyl moieties to BSA is one of the following: 7.0-7.1; 7.1-7.2; 7.2-7.3; 7.3-7.4; 7.4-7.5; 7.5-7.6; 7.6-7.7; 7.7-7.8; 7.8-7.9; 7.9-8.0; 8.0-8.1; 8.1-8.2; 8.2-8.3; 8.3-8.4; 8.4-8.5; 8.5-8.6; 8.6-8.7; 8.7-8.8; 8.8-8.9; 8.9-9.0; 9.0-9.1; 9.1-9.2; 9.2-9.3; 9.3-9.4; 9.4-9.5; 9.5-9.6; 9.6-9.7; 9.7-9.8; 9.8-9.9; or 9.9-10.0; 7.0-7.2; 7.2-7.4; 7.4-7.6; 7.6-7.8; 7.8-8.0; 8.0-8.2; 8.2-8.4; 8.4-8.6; 8.6-8.8; 8.8-9.0; 9.0-9.2; 9.2-9.4; 9.4-9.6; 9.6-9.8; or 9.8-10.0.

Adjuvants suitable for use in the third pharmaceutical composition are known. In a preferred embodiment, the suitable adjuvant is aluminum hydroxide gel. Similarly, buffers suitable for use in the third pharmaceutical composition are known. In a preferred embodiment, the suitable buffer is phosphate buffered saline.

In a preferred embodiment, the third pharmaceutical composition comprises a plurality of ethanol-succinyl-BSA, wherein (i) the average ratio of ethanol-succinyl moieties to BSA is at least 7.5; (ii) the composition is free of EDAC (and preferably is free of any amidine); (iii) the ethanol-succinyl-BSA is adsorbed onto aluminum hydroxide gel; and (iv) the ethanol-succinyl-BSA so adsorbed is suspended in phosphate buffered saline.

This invention further provides a method (“third therapeutic method”) for treating a subject addicted to alcohol comprising administering to the subject an effective amount of the third pharmaceutical composition (i.e., a therapeutically effective amount). Preferably, the subject is human.

In the third therapeutic method, the third pharmaceutical composition can be administered according to any appropriate dosing regimen. In one embodiment, the third pharmaceutical composition is administered via a single dose. In a preferred embodiment, the composition is administered via a plurality of doses, such as two doses 30 days apart. In a further preferred embodiment, the composition is administered via three doses at 30-day intervals (i.e., at day 0, day 30 and day 60). In yet another embodiment, this administration is followed with one or more “boosters” at suitable intervals (e.g., every two or three years).

In a preferred embodiment of the third therapeutic method, the method comprises injecting the subject with an effective amount of the first pharmaceutical composition, wherein the composition is administered via three doses at 30-day intervals. Preferably, the subject is human.

This invention also provides a method (“third prophylactic method”) for inhibiting the onset of alcohol addiction in a non-addicted subject comprising administering to the subject an effective amount of the third composition (i.e., a prophylactically effect amount). Preferably, the subject is human.

In the third prophylactic method, the third pharmaceutical composition can be administered according to any appropriate dosing regimen. In one embodiment, the third pharmaceutical composition is administered via a single dose. In a preferred embodiment, the composition is administered via a plurality of doses, such as two doses 30 days apart. In a further preferred embodiment, the composition is administered via three doses at 30-day intervals (i.e., at day 0, day 30 and day 60). In yet another embodiment, this administration is followed with one or more “boosters” at suitable intervals (e.g., every two or three years).

In a preferred embodiment of the third prophylactic method, the method comprises injecting the human subject with an effective amount of the third prophylactic composition, wherein the composition is administered via three doses at 30-day intervals.

The third composition (as opposed to the third pharmaceutical composition) is also envisioned, mutatis mutandis, for use in the present therapeutic and prophylactic methods.

This invention provides a first synthetic method for making ethanol-succinyl-BSA, comprising the following steps:
(a) contacting alcohol with succinic anhydride in the presence of benzene under conditions permitting the formation of ethanol-succinate; and
(b) contacting the resulting ethanol-succinate with BSA under conditions permitting the formation of ethanol-succinyl-BSA, wherein (i) such conditions comprise the presence of tributylamine, dioxane, and isobutyl chloroformate, and (ii) neither step (a) nor step (b) is performed in the presence of EDAC.

In a preferred embodiment of the first synthetic method, neither step (a) nor step (b) is performed in the presence of an amidine.

This invention provides a second synthetic method for making ethanol-succinyl-BSA, comprising contacting ethanol-succinate with BSA under conditions permitting the formation of ethanol-succinyl-BSA, wherein (i) such conditions comprise the presence of tributylamine, dioxane, and isobutyl chloroformate, and (ii) the contacting is performed in the absence of EDAC.

In a preferred embodiment of the second synthetic method, the contacting is performed in the absence of any amidine.

This invention provides fifth and sixth articles of manufacture (also referred to as kits). The fifth article comprises (a) a composition of matter comprising a plurality of ethanol-succinyl-BSA, wherein (i) the average ratio of ethanol-succinyl moieties to BSA is at least 7.0; (ii) the composition is free of EDAC; (iii) the ethanol-succinyl-BSA is adsorbed onto a suitable adjuvant; and (iv) the ethanol-succinyl-BSA so adsorbed is suspended in a suitable buffer, and (b) a label indicating a use for the composition in treating a human subject addicted to alcohol.

In a preferred embodiment, the fifth article comprises (a) a composition of matter comprising a plurality of ethanol-succinyl-BSA, wherein (i) the average ratio of ethanol-succinyl moieties to BSA is at least 7.5; (ii) the composition is free of EDAC; (iii) the ethanol-succinyl-BSA is adsorbed onto aluminum hydroxide gel; and (iv) the ethanol-succinyl-BSA so adsorbed is suspended in phosphate buffered saline, and (b) a label indicating a use for the composition in treating a human subject addicted to alcohol.

The sixth article comprises (a) a composition of matter comprising a plurality of ethanol-succinyl-BSA, wherein (i) the average ratio of ethanol-succinyl moieties to BSA is at least 7.0; (ii) the composition is free of EDAC; (iii) the ethanol-succinyl-BSA is adsorbed onto a suitable adjuvant; and (iv) the ethanol-succinyl-BSA so adsorbed is suspended in a suitable buffer, and (b) a label indicating a use for the composition in inhibiting the onset of alcohol addiction in a non-addicted human subject.

In a preferred embodiment, the sixth article comprises (a) a composition of matter comprising a plurality of ethanol-succinyl-BSA, wherein (i) the average ratio of ethanol-succinyl moieties to BSA is at least 7.5; (ii) the composition is free of EDAC; (iii) the ethanol-succinyl-BSA is adsorbed onto aluminum hydroxide gel; and (iv) the ethanol-succinyl-BSA so adsorbed is suspended in phosphate buffered saline, and (b) a label indicating a use for the composition in inhibiting the onset of alcohol addiction in a non-addicted human subject.

Preferably, in the fifth and sixth articles, the composition is free of any amidine.

This invention will be better understood by reference to the examples which follow, but those skilled in the art will readily appreciate that the specific examples detailed are only illustrative of the invention as described more fully in the claims which follow thereafter.

### EXAMPLES

### Example 1

### Production of Pure Morphine

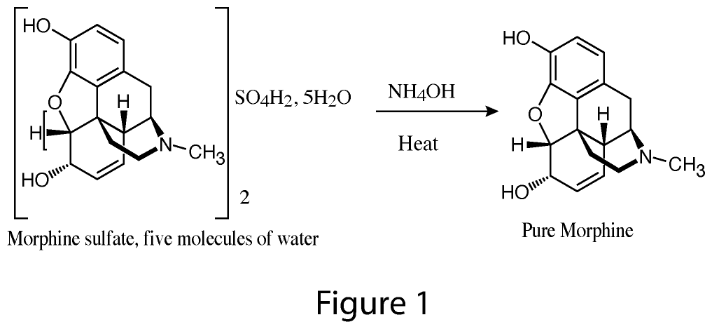

Morphine sulfate (formula 1 in FIG. 1) and ammonium hydroxide were purchased respectively from Tamad Pharmaceutical Company (Iran) and Merck (Germany). 0.0179 mol of morphine sulfate was dissolved in distilled water at room temperature and the solution was brought to pH 7 with ammonium hydroxide. The pure morphine molecules precipitate in suspension. The precipitate was extracted with a Buchner funnel, distilled with distilled water, and dried at room temperature under vacuum over P₂O₅, yielding 9.122 g of pure morphine. The efficiency by molecular weight was 76.02% (formula 2 in FIG. 1).

### Example 2

### Production of Morphine-6-Succinate (M-6-S)

The reaction of morphine with succinic anhydride in benzene leads to the selective succinylation of the 6-hydroxyl morphine group.

Specifically, 0.035 mol morphine with 0.1 mol succinic anhydride in benzene was poured into a 1-liter double-spouted balloon, to which was added 0.1 mol succinic anhydride again under refrigerant after two to three hours of boiling.

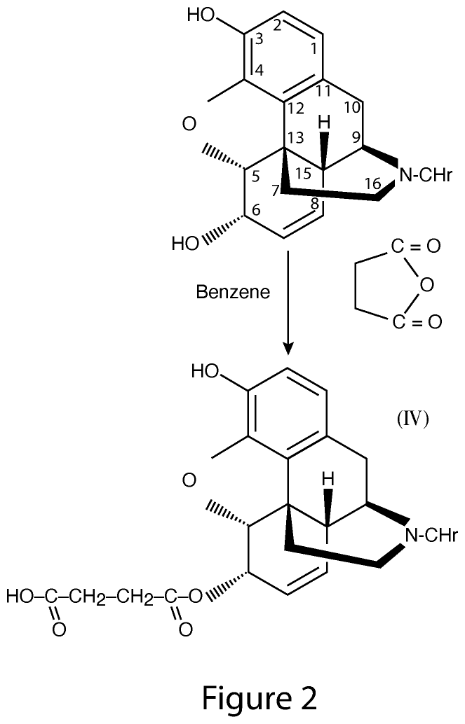

The reaction mixture was cooled to room temperature, benzene was slowly removed, and the residual benzene evaporated with the help of nitrogen gas flow. After the mixture inside the balloon was completely benzene-free, the residue was mixed in 1,000 ml of distilled water, its pH was brought to 5 with HCl, it was filtered with a Buchner funnel with succinic anhydride, and was filtered again. The resulting paste was a greenish-green solution of morphine-6-succinate. The pH of the filter solution was then raised to 8.5 and the solution was filtered again to remove morphine from the reaction. The pH of the morphine-6-succinate (M-6-S) filtrate solution was then adjusted to 7 and refrigerated overnight to crystallize the M-6-S. The crystals were separated by Buchner funnel filtration in the presence of anhydrous CaCl₂ in vacuo at 60° C. in a lactic desiccator. Crystallized morphine-6-succinate (which is white and needle-shaped) was stored in brown closed vials. The efficiency of this process was 70%-75%. See FIG. 2.

### Example 3

### Production of Morphine-6-Succinyl-BSA (M-6-S-BSA)

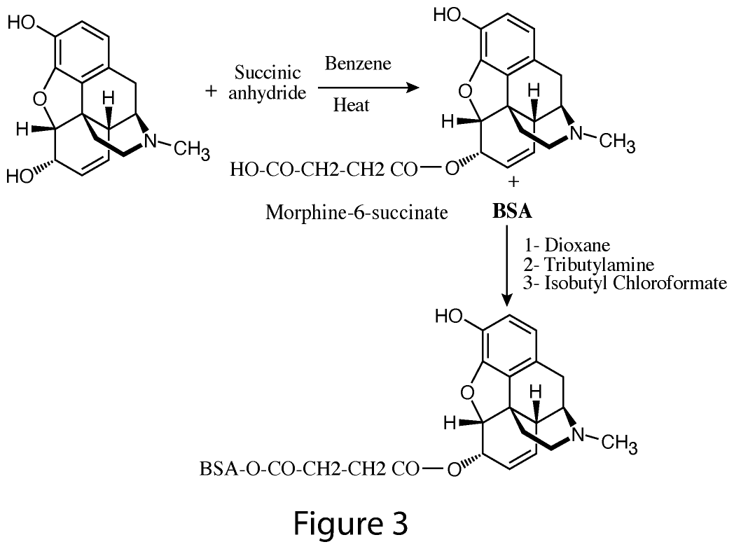

Tri-N-butyl amine, dioxane, isobutyl chloroformate and sodium hydroxide were purchased from Merck (Germany). In a three-liter one-way balloon, 2.15 mM morphine-6-succinate and 2.53 mM Trim-N-butyl amine were mixed in 21.5 mL dioxane. After the mixture was brought to 10° C., 2.55 mM isobutyl chloroformate was added. The reaction was continued at room temperature for 60 minutes. The mixture was then immediately dissolved in a 5° C. cold solution of 0.051 mmol BSA (dissolved in 186 mL 1: 1 water-dioxane; spectral grade) and 3.58 mL normal NaOH while mixing well. Following the addition of the solution, the color became slightly opaque and gas bubbles appeared. The pH of the solution was then adjusted to 7.5 with normal NaOH, After one hour with NaOH, the pH of the solution was adjusted to 7, and mixing continued at 5° C. for more 2 hours. The product was poured into a dialysis bag with a cuff of 30,000 Daltons and dialyzed overnight under low-flow urban (tap) water, Then, in a beaker, the pH was adjusted to 4.5 with HCl. Morphine-6-succinyl-BSA began to precipitate. The precipitate was kept inside the beaker at 4° C. for one night, and then separated via centrifuge the next day. The precipitate of morphine-6-succinyl-BSA was suspended in 100 ml of distilled water and brought to pH 7 with 0,1 NaHCO3 solution. Then, the product was dialyzed under tap water for 4 hours. The yield was 2.29 g (75-70% efficiency). 45 doses of antigen were produced. The product was dissolved in pyrogen-free distilled water, and filtration and sterilization continued. Ten doses were used for quality control, waste management, and expiration. See FIG. 3.

### Example 4

### Morphine Vaccine Formulation (M-6-S-BSA)

| | | Formulation values | |
|---|---|---|---|
| Components | The role of | of one | |
| Formulation | each component | dose per ml | |
| Antigenic protein (M-6-S- | Immunogen | 50 | ug |
| BSA) | | | |
| Aluminum hydroxide | Adjuvant | 0.5 | mg |
| Sodium chloride | Buffer | 8 | mg |
| Anhydrous disodium | Buffer | 1.12 | mg |
| hydrogen phosphate | | | |
| Sodium dihydrogen | Buffer | 1.1 | mg |
| phosphate monohydrate | | | |
| Injectable distilled water Qs | Solvent | 1 | ml |

### Example 5

### Production of Heroin-6-Succinyl-BSA (H-6-S-BSA)

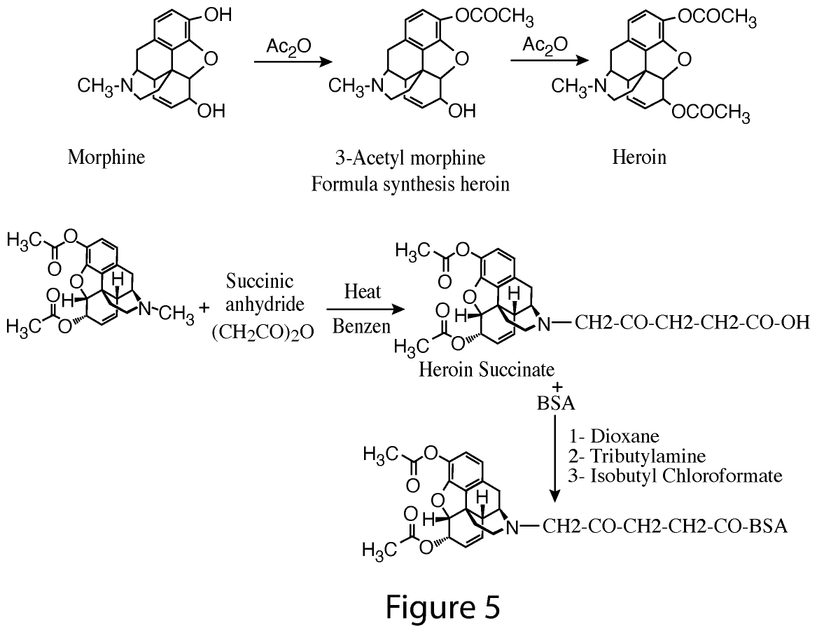

The reaction of heroin with succinic anhydride in benzene yields heroin-6-succinate. Heroin-6-succinate is reacted with BSA (i.e., BSA-(CH₂)₄-NH₂), tributyl amine, dioxane, and isobutyl chloroformate to yield heroin-6-succinyl-BSA (also referred to as BSA-heroin-6-succinate). See FIG. 5. The reaction conditions used in the production of morphine-6-succinyl-BSA in Examples 2 and 3 above can also be used to produce heroin-6-succinyl-BSA.

### Example 6

### Heroin Vaccine Formulation (H-6-S-BSA)

| | | Formulation values | |
|---|---|---|---|
| Components | The role of | of one | |
| Formulation | each component | dose per ml | |
| Antigenic protein (H-6-S- | Immunogen | 50 | ug |
| BSA) | | | |
| Aluminum hydroxide | Adjuvant | 0.5 | mg |
| Sodium chloride | Buffer | 8 | mg |
| Anhydrous disodium | Buffer | 1.12 | mg |
| hydrogen phosphate | | | |
| Sodium dihydrogen | Buffer | 1.1 | mg |
| phosphate monohydrate | | | |
| Injectable distilled water Qs | Solvent | 1 | ml |

### Example 7

### Production of Ethanol-Succinyl-BSA (E-S-BSA)

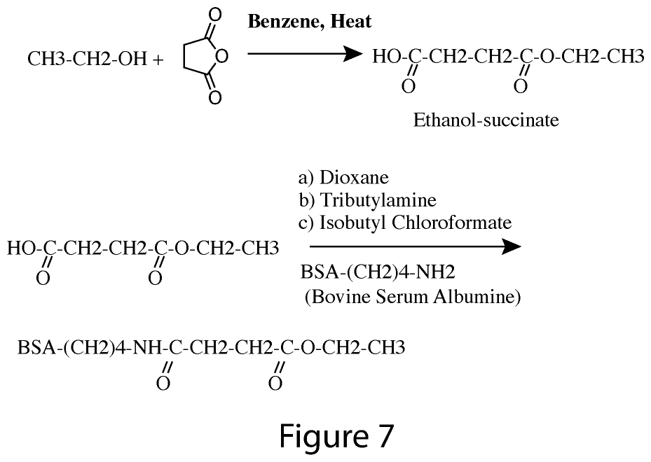

The reaction of ethanol with succinic anhydride in benzene yields ethanol succinate. Ethanol succinate is reacted with BSA (i.e., BSA-(CH₂)₄-NH₂), tributyl amine, dioxane, and isobutyl chloroformate to yield ethanol-succinyl-BSA (also referred to as BSA-ethanol-succinate). See FIG. 7. The reaction conditions used in the production of morphine-6-succinyl-BSA in Examples 2 and 3 above can also be used to produce ethanol-succinyl-BSA.

### Example 8

### Alcohol Vaccine Formulation (E-S-BSA)

The following table sets forth the contents of a 1 ml dosage form (vial) of the subject alcohol vaccine (i.e., ethanol-succinyl-BSA).

| | | Formulation values | |
|---|---|---|---|
| Components | The role of | of one | |
| Formulation | each component | dose per ml | |
| Antigenic protein (E-S-BSA) | Immunogen | 50 | ug |
| Aluminum hydroxide | Adjuvant | 0.5 | mg |
| Sodium chloride | Buffer | 8 | mg |
| Anhydrous disodium | Buffer | 1.12 | mg |
| hydrogen phosphate | | | |
| Sodium dihydrogen | Buffer | 1.1 | mg |
| phosphate monohydrate | | | |
| Injectable distilled water Qs | Solvent | 1 | ml |

### REFERENCES

1 A. Akbarzadeh, et al., Asian J. of Biochemistry, 2(1):58-65 (2007).

2. A. Akbarzadeh, et al., J. of Pharm. And Toxicol., 4(1):3035 (2009).

3. A. Akbarzadeh, et al., Biotechnology and Applied Biochem., (Dec. 23, 2010).

4. F. Li, et al., Org. Biomol. Chem. 2014 Oct 7; 12(37): 7211-7232.

5. M. Pravetoni, et al., Biochem. Pharmacol. 2012 Feb 15;83(4):543-50.

6. M. Pravetoni, et al., J. Pharmacol. Exp. Ther. 2012 Apr;341(1):225-32.

7 Y. Hu, et al., Hum. Vaccin. Immunother. 2014;10(1):64-72.

1. A composition of matter comprising a plurality of morphine-6-succinyl-bovine serum albumin (BSA), wherein the average ratio of morphine-6-succinyl moieties to BSA is at least 7.0.

2. The composition of claim 1, wherein the composition is free of 1-ethyl-3-(3-dimethylaminopropyl) carbodiimide (EDAC) and free of any amidine.

3. (canceled)

4. The composition of claim 1, wherein the average ratio of morphine-6-succinyl moieties to BSA is selected from the group consisting of at least 7.5, at least 8.0, and at least 8.2.

5. A composition of matter comprising a plurality of morphine-6-succinyl-BSA, wherein (i) the average ratio of morphine-6-succinyl moieties to BSA is at least 8.2; and (ii) the composition is free of EDAC and free of any amidine.

6. (canceled)

7. A composition of matter comprising a plurality of morphine-6-succinyl-BSA, wherein (i) the average ratio of morphine-6-succinyl moieties to BSA is at least 7.0; (ii) the composition is free of EDAC and free of any amidine; (iii) the morphine-6-succinyl-BSA is adsorbed onto a suitable adjuvant; and (iv) the morphine-6-succinyl-BSA so adsorbed is suspended in a suitable buffer.

8. (canceled)

9. The composition of claim 7, wherein the average ratio of morphine-6-succinyl moieties to BSA is selected from the group consisting of at least 7.5, at least 8.0, and at least 8.2.

10. The composition of claim 7, wherein the suitable adjuvant is aluminum hydroxide gel.

11. The composition of claim 7, wherein the suitable buffer is phosphate buffered saline.

12. The composition of claim 7, wherein the composition comprises a plurality of morphine-6-succinyl-BSA, wherein (i) the average ratio of morphine-6-succinyl moieties to BSA is at least 8.2; (ii) the composition is free of EDAC and free of any amidine; (iii) the morphine-6-succinyl-BSA is adsorbed onto aluminum hydroxide gel; and (iv) the morphine-6-succinyl-BSA so adsorbed is suspended in phosphate buffered saline.

13.-25. (canceled)

26. A method for making morphine-6-succinyl-BSA, comprising the following steps:
(a) contacting morphine with succinic anhydride in the presence of benzene under conditions permitting the formation of morphine-6-succinate; and
(b) contacting the resulting morphine-6-succinate with BSA under conditions permitting the formation of morphine-6-succinyl-BSA, wherein (i) such conditions comprise the presence of tributylamine, dioxane, and isobutyl chloroformate, and (ii) neither step (a) nor step (b) is performed in the presence of EDAC or in the presence of any amidine.

27. (canceled)

28. A method for making morphine-6-succinyl-BSA, comprising contacting morphine-6-succinate with BSA under conditions permitting the formation of morphine-6-succinyl-BSA, wherein (i) such conditions comprise the presence of tributylamine, dioxane, and isobutyl chloroformate, and (ii) the contacting is performed in the absence of EDAC and in the absence of any amidine.

29. (canceled)

30. An article of manufacture comprising (a) the composition of claim 7, and (b) a label indicating a use for the composition in treating a human subject addicted to morphine.

31. An article of manufacture comprising (a) the composition of claim 12, and (b) a label indicating a use for the composition in treating a human subject addicted to morphine.

32. An article of manufacture comprising (a) the composition of claim 7, and (b) a label indicating a use for the composition in inhibiting the onset of morphine addiction in a non-addicted human subject.

33. An article of manufacture comprising (a) the composition of claim 12, and (b) a label indicating a use for the composition in inhibiting the onset of morphine addiction in a non-addicted human subject.

34.-102. (canceled)
